(19) United States
(12) Patent Application Publication
(10) Pub. No.: US 2011/0224904 A1
(43) Pub. Date: Sep. 15, 2011

(54) METHOD FOR MONITORING THE SPATIAL ENVIRONMENT OF A MOBILE DEVICE

(76) Inventors: Wendelin Feiten, Neubiberg (DE); Thomas Redel, Poxdorf (DE); Raoul Daniel Zöllner, Heilbronn (DE)

(21) Appl. No.: 13/043,553

(22) Filed: Mar. 9, 2011

(30) Foreign Application Priority Data
Mar. 10, 2010 (DE) 10 2010 010 875.8

(51) Int. Cl. G06F 17/00; A61B 6/08

(52) U.S. Cl. 701/301; 378/205

(57) ABSTRACT

A method for monitoring a spatial environment of a mobile device is provided. During the movement of the device along a predefined path, a three-dimensional spatial region of the spatial environment is captured by a detection device. A three-dimensional environment model is created and/or updated from the captured spatial region at cyclical intervals and is specified by spatial volumes in the spatial region occupied by objects. Actions of the device for preventing a collision with the objects and a risk of collision are determined. One of the actions is then performed if the risk of collision exceeds a predefined value. The method has the advantage that actions for collision avoidance are calculated preemptively at cyclical intervals, so that one of the actions can be performed with a short latency period in an impending collision, that is if the risk of collision exceeds the predefined value.

### CROSS REFERENCE TO RELATED APPLICATIONS

This application claims priority of German application No. 10 2010 010 875.8 filed Mar. 10, 2010, which is incorporated by reference herein in its entirety.

### FIELD OF THE INVENTION

The invention relates to a method for monitoring the spatial environment of a mobile device, in particular of a medical device, and a corresponding monitoring device.

### BACKGROUND OF THE INVENTION

In the field of robotics many different methods are known, with which the movement of a robot can be planned interactively, where by capturing the environment of the robot with corresponding sensors, a collision of the robot with other objects can be avoided by means of the automatic execution of a braking or avoidance movement.

The publication [1] describes a detection apparatus for monitoring of the environment of a medical device in the form of a light curtain capable of variable positioning, which can be realized by means of a laser scanner.

The document [2] describes the monitoring of a medical device based on an array of direct distance-measuring sensors.

The document [3] discloses motion planning for a medical device, in which the risk of collision in the case of movement along a path is taken into account in a suitable manner.

In the document [4] the monitoring of the spatial environment of a medical device is described with detection means both arranged on the device and fixed in the room.

The above publications [1] to [4] disclose in part the performing of a stopping or avoidance movement for the prevention of a collision, without examining the nature of the calculation of the stopping or avoidance movement.

Publication [5] describes the motion planning for a robot arm and discloses in detail corresponding methods, such as how collision-free paths and corresponding avoidance movements for the prevention of collisions can be determined.

The known method for monitoring the movement of a mobile device is prone to the problem that the execution of a corresponding stopping or avoidance movement of the device upon the incidence of a danger of collision is connected with a time delay, as the stopping or avoidance movement must first be determined via time-consuming computer-aided methods.

### SUMMARY OF THE INVENTION

It is thus the object of the invention to create monitoring of the spatial environment of a mobile device, in which actions for the prevention of collisions in the case of a danger of collision are performed with a minimal time delay by the device.

This problem is solved by the method or the device according to the independent claims. Developments of the invention are defined in the dependent claims.

In the inventive monitoring method, which in particular serves the monitoring of a medical device, a three-dimensional spatial region of the spatial environment of the device is captured during the movement of the device along a predefined path by means of one or more detection means.

Here the following steps are performed in the cyclical periods at a particular point in time:
a) A three-dimensional environment model is created and/or updated from the captured spatial region, where the environment model specifies spatial volumes in the spatial region occupied by one or more objects.
b) One or more actions of the device for the prevention of a collision with the object or objects and a risk of collision for the collision are determined based on the environment model and the predefined path of the model, where one of the actions is then performed if the risk of collision exceeds a predefined value. The risk of collision can for example be determined via the distance between device and the objects, where the smaller the distance involved, the greater the risk of collision.

The inventive method is characterized in that the determining of an action for collision avoidance is no longer coupled to the actual incidence of a danger of collision. Rather, possible actions for the prevention of a collision are determined continuously at cyclical intervals. These actions are held available and one of the actions is only then performed if a danger of collision actually arises, that is if a determined risk of collision exceeds a predefined value. In this way it is avoided that only upon the incidence of the danger of collision does the calculation of an action for collision avoidance takes place, but rather that reference back to an action already previously determined takes place, so that the action for collision avoidance can be performed more rapidly. Corresponding methods for determining actions for collision avoidance are here known per se from the prior art. In this connection attention is drawn in particular to the disclosure of publication [5].

In a particularly preferred embodiment of the inventive method, in step b) the maneuvering room of the device is modeled with the aid of the environment model, the spatial volumes in the captured spatial region specified, in which the device can move safe from collision, where based on the maneuvering room and a prescribed dynamic model of the possible movements of the device the action or actions is/are determined. The prescribed dynamic model here represents the dynamic outline conditions for the movement of the device in the form of realizable accelerations and directions of motion.

In a particularly preferred embodiment of the inventive method the actions for collision avoidance specify stopping and/or avoidance movements of the device. In this way, different strategies for collision avoidance are realized.

In a further embodiment of the inventive method, in step b) a risk of damage upon execution of the action is determined for each of the determined actions, where that action with the lowest risk of damage is performed if the risk of collision exceeds the predefined value. An optimization strategy for selection of the most suitable action is hereby arrived at in a suitable manner. The risk of damage can here be defined or categorized in a different manner, and describes a quantitative and/or qualitative degree of damage, which is assigned to the corresponding action. The risk of damage can for example be determined depending upon whether the respective action causes personal injury or material damage, where personal injury leads to a higher risk of damage. If all possible actions for collision avoidance have a risk of damage of zero or the same risk of damage, still further optimization objectives can be implemented, for example that action can be performed, which has the shortest braking distance.

In a further embodiment of the inventive method the action or actions at are least partially determined taking account of a prediction of the movement of the object or objects. In this way in particular, a strategy for collision avoidance can be planned in a preemptive manner. The prediction of the movement is for example laid down in a suitable manner depending upon the initial detection of an object in the monitored spatial region. If the mobile device is for example an X-ray system in the form of a C-arm, which is explained in greater detail below, it is possible to predict that upon the appearance of a object from the inner area of the C-arm in a monitored spatial region at the forward ends of the C-arm it is indicated that the object will enter further into the monitored spatial region, as it is assumed that the detected object takes the form of a just commenced movement of a body part of the patient being examined.

In a further embodiment of the inventive method the spatial region is at least partially captured by one or more detection means arranged on the device and moving with the latter. In certain circumstances, however, the possibility exists that the spatial region is at least partially captured by means of one or more fixed arranged detection means. In the inventive method, active distance-measuring means are preferably used as detection means, for example one or more active distance-measuring 3D-cameras and/or one or more pivotable laser scanner. Active or direct distance-measuring detection means is here to be understood as such detection means as actively transmit a signal and then receive it again, where the distance to objects is determined from the change in the signal as a result of reflection or scattering from objects or from the transit time of the transmitted and again received signal. The signal can here be embodied as desired, and can in particular take the form of electromagnetic waves (for example light in the visible or not-visible range) or sound waves (in particular ultrasound waves).

In a further embodiment of the inventive method, spatial volumes occupied by the device are specified in the determined environment model in addition to spatial volumes occupied by one or more objects. Such spatial volumes can for example be determined with fixedly mounted detection means.

As already mentioned above, a particularly preferred area of application of the inventive method is the monitoring of a mobile device in the form of a medical device. Here, the patient to be examined or treated with the medical device and/or one or more further persons and/or objects are preferably captured with the detection means in the monitored spatial region and specified as objects in the environment model. The mobile device can here be an X-ray system and in particular the abovementioned C-arm. If the device is a C-arm, the detection means is/are preferably attached to this arm in such away that a spatial region within the C-arm and/or a spatial region at the opposite ends of the limbs of the C-arm and/or a spatial region in a spatial direction perpendicular to the plane of the C-arm are captured.

In addition to the above-described method, the invention further relates to a device for monitoring the spatial environment of a mobile device, in particular of a medical device, comprising one or more detection means, with which during the movement of the device along a predefined path a three-dimensional spatial region of the spatial environment is captured, where the device further comprises an analysis means, which creates and/or updates a three-dimensional environment model from the captured spatial region at cyclical intervals with a first means, where the environment model specifies spatial volumes in the spatial region occupied by one or more objects. At cyclical intervals, the analysis means further determines, with a second means, one or more actions of the device for the prevention of a collision with the object or objects and a risk of collision for the collision based on the environment model and the predefined path of the device, where one of the actions is then performed if the risk of collision exceeds a predefined value. If appropriate, the first and/or the second means can here contain further means.

The inventive device is preferably embodied in such a way that it has one or more means of for performing the above-described advantageous embodiments of the inventive method.

### BRIEF DESCRIPTION OF THE DRAWINGS

Exemplary embodiments of the invention are described in detail below, on the basis of the attached figures, in which:

FIG. 1 shows a schematic representation of a device for performing an embodiment of the inventive method; and

FIG. 2 to FIG. 5 show schematic representations of scenarios for collision avoidance between a C-arm and a patient according to embodiments of the inventive method.

### DETAILED DESCRIPTION OF THE INVENTION

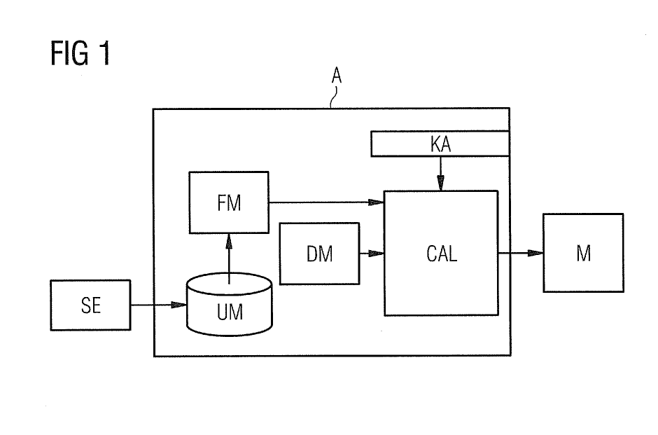

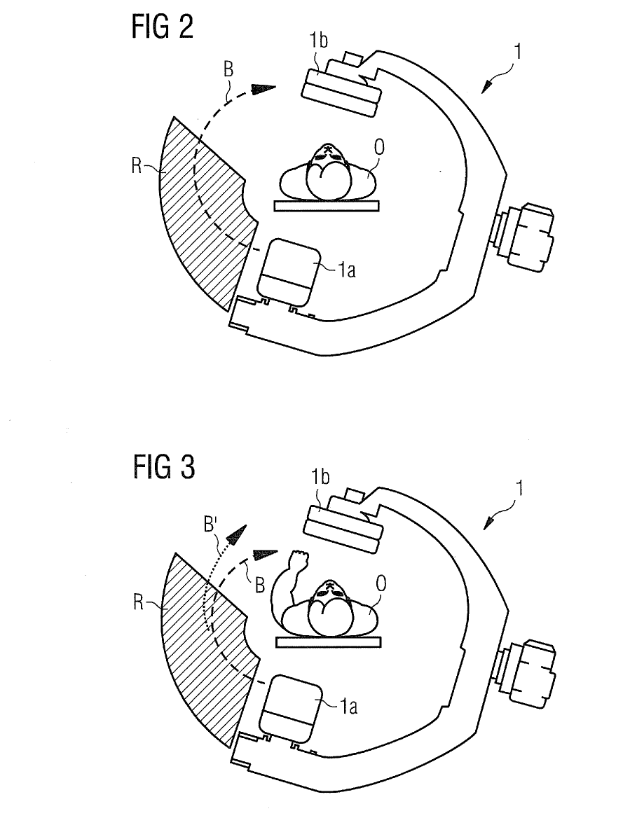

FIG. 1 shows, in a schematic representation, a device for performing an embodiment of the inventive method. The device enables the monitoring of the spatial region of a medical device, which is identified with the reference character M in FIG. 1. One embodiment of such a medical device M in the form of a C-arm is described further below with reference to FIGS. 2 to 5. The steps of the inventive method are performed in a computer-aided manner via analysis unit A, which analyzes the data from a sensor system SE, which monitors the spatial region around the medical device M, and based on this, controls the movement of the medical device such that collisions between the device and objects captured in the monitored environment are avoided.

The medical device M moves on a predefined path, which is known to the analysis unit A, and the sensor data captured during this path movement is initially fed to a first module of the analysis unit A, which determines an incremental environment model or environmental model UM cyclically, at regular intervals. The incremental environmental model is here based on existing model knowledge (for example device equipment and furniture in the treatment room) and is built up by the data from the sensor system SE and updated on a chronological cycle through the fusion of new measured values. The internal representation here corresponds to a volume model with variable granularity, depending on the resolution of the sensors employed. In the volume model the objects captured by the sensor system are specified as spatial volumes. In a preferred variant the sensor system. SE captures the space of the C-arm located in the direction of movement in the case of the monitoring of a C-arm, where sensors suitable for this purpose, such as for example active distance-measuring sensors in the form of 3D-cameras or laser scanners, are used. In certain circumstances, it is also possible to use additional further sensors for monitoring of the environment, such as for example fixedly mounted sensors, for example sensors fixed to the ceiling of the treatment room, which do not move along with the C-arm.

After the determining or updating of the incremental environmental model UM, a maneuvering room modeling FM is performed in a further module based on this model, which specifies the spatial volumes in the spatial region captured by the sensor system, in which the medical device M can move safe from collision. In an analogous manner to the incremental environmental model UM, the maneuvering room of the system is also calculated at cyclical intervals and represented in a Cartesian manner as a volume model. Via a cinematic or dynamic modeling of the medical device, which takes into account the predefined trajectory of the movement of the device and cinematic and dynamic outline conditions of the movements, possible stopping or avoidance strategies for the prevention of collisions with the objects captured by the sensor system are then determined in a calculation model CAL using the modeled maneuvering room.

A cyclical collision analysis is in turn performed in a module KA, where the collision analysis delivers as its output value a corresponding risk of collision relating to the collision of the medical device with the captured objects in the environment of the device. It is here significant for the invention that independently of the risk of collision, possible stopping or avoidance strategies are always determined at cyclical intervals, even if the risk of collision is very low or a risk of collision of zero is determined. If the risk of collision lies below a predefined value, the determining of the avoidance or stopping strategies in the module CAL has no consequences for the controlling of the medical device M. The stopping or avoidance strategies are however held available in the module CAL. If a risk of collision which exceeds a predefined value should be determined via the module KA, the performing of a corresponding avoidance or stopping procedure ultimately takes place based on the avoidance or stopping movements previously calculated and held available.

If a number of avoidance or stopping strategies are stored in the module CAL, a suitable optimization strategy for selecting the movement most suitable for collision avoidance is further employed. The optimization may depend on any criteria, for example on a risk of damage, which may be connected with a corresponding stopping or avoidance procedure. This risk of damage can likewise be calculated in the module CAL in a suitable manner. It represents a value which expresses whether or with what degree of likelihood damage to objects or persons will arise during a stopping or avoidance procedure, where personal injury leads to a higher risk of damage than damage to objects. Should only avoidance or stopping movements have been determined which result in no damage, further optimization objectives can be taken into account, for example from the multiplicity of movements calculated, that stopping or avoidance movement can be used which has the shortest stopping distance.

With the embodiment shown in FIG. 1 a motion control is thus realized, which in each step at cyclical intervals calculates a maximum braking acceleration for a stopping or avoidance procedure for each axis of movement of the device, so that in the case of a collision event, that is upon the exceeding of a predefined risk of collision, these braking accelerations can be passed directly to the drive mechanisms of the device. As already mentioned, suitable optimization strategies are realized within the framework of this calculation which decide, at cyclical intervals and based on the overall system, which avoidance or stopping movement from the quantity of calculated movements is most suitable.

The system for monitoring of a mobile device and an associated collision avoidance with objects in the environment of the device described on the basis of FIG. 1 has a number of advantages. In particular the latency periods of the system are reduced by the calculation of corresponding stopping or avoidance movements performed at cyclical intervals. Furthermore, the best possible solution for collision avoidance is made available in each situation with minimal time delay. In addition, through the use of suitable optimization strategies, an optimum stopping or avoidance movement is performed by the device with a view to minimizing risk.

FIG. 2 to FIG. 5 described below show the implementation of an inventive collision avoidance based on an X-ray system in the form of a so-called C-arm. FIG. 2 here shows a side view of the C-arm during the performing of an examination of an object O in the form of a patient. In FIG. 2 to FIG. 5 the C-arm is identified with reference number 1 and represents a device for X-raying patients, which is known per se. The arm comprises an X-ray emitter 1a at one end of the limb of the C-arm and a corresponding detector 1b at the other end of the limb of the C-arm. The C-aim can here be moved about a multiplicity of axes in a suitable manner, in order hereby to X-ray the organs of a patient O. In the following FIG. 2 to FIG. 5, during the examination of the patient the C-arm O describes a circular movement in the blade plane. This movement is indicated by a curved dashed path B. This path represents the planned movement of the C-arm according to FIG. 2.

The segment of a circle between the forward ends of the C-arm representing the extension of the C-arm is monitored with corresponding detection means, such as for example active distance-measuring cameras or laser scanners. In FIG. 2 only a section R of this segment of a circle is indicated, which is characterized in that the risk of collision upon intrusion of the object O into this section exceeds a predefined value for the path movement B represented, and leads to the triggering of a stopping or avoidance procedure. On the other hand the risk of collision upon intrusion of the object O into a section of the segment outside the area R is still so low that no stopping or avoidance procedure is triggered. However according to the invention a suitable stopping or avoidance strategy is determined and held available even in those cases in which the risk of collision remains low. In the scenario shown in FIG. 2, no object is detected in the segment of a circle between the limbs of the C-arm, so that no avoidance or stopping strategy is planned. The movement thus proceeds along the planned path B.

In the scenario in FIG. 3 the patient O raises his or her left aim, so that this arm is captured as an object via the detection means in the segment of a circle between the two limbs of the C-arm. As a result, the steps set out in FIG. 1 are performed, that is a stopping or avoidance movement is determined at cyclical intervals and the risk of collision of the C-arm with the object O ascertained. In the situation depicted in FIG. 3, the risk of collision is so low that the C-arm still needs to perform no stopping or avoidance movement. Nevertheless, an avoidance path B′ indicated by a dotted arrow is already determined. The avoidance path is here updated at cyclical intervals during the movement of the C-arm and held available in a corresponding memory.

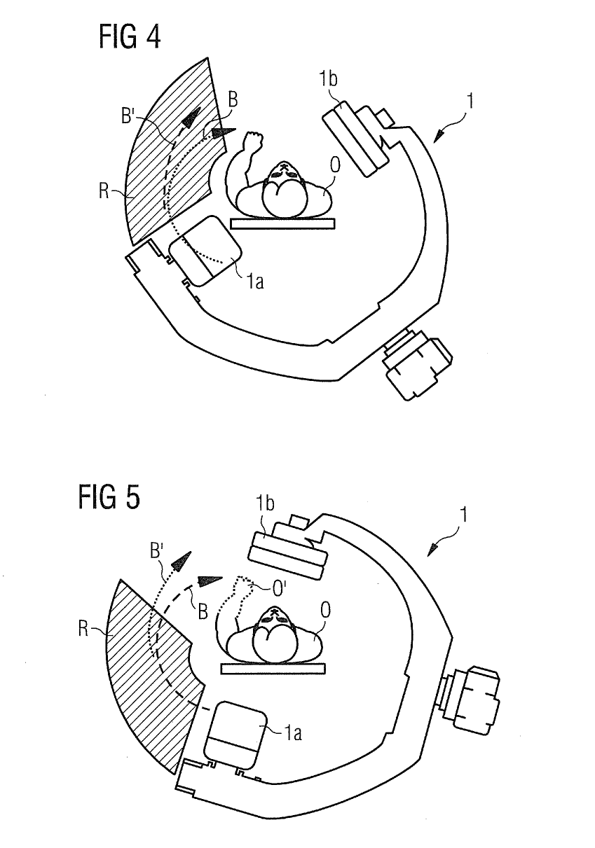

FIG. 4 shows the situation, in which the further movement of the C-arm with the outstretched arm of the patient O will now lead to an actual risk situation, which arises as a result of the arm encroaching into spatial section R, which is synonymous with a risk of collision determined at cyclical intervals exceeding a predefined value. This has the result that from now on the avoidance movement along the path B′ held available is actually performed, indicated here by the path B′ now being represented by a dashed arrow, whereas the path B is represented by a dotted arrow. A movement along the path B′ thus takes place, and the original circular path is discarded. According to the invention the avoidance movement can be implemented with a very short latency period, as an avoidance movement has previously already been determined on a cyclical basis.

FIG. 5 shows a further variant of the inventive method for collision avoidance based on the circular movement of the C-arm 1 along path B. The variant in FIG. 5 differs from the embodiment according to FIG. 2 to FIG. 4 in that upon the calculation of an avoidance or stopping movement, a commenced movement of the patient O is further predicted. FIG. 5 here shows the situation in which the patient is just beginning to extend his or her arm. This event, which is captured in that an object is moving outwards from the interior of the C-arm, leads to the cyclical determining of a stopping or avoidance movement based on the prediction that the arm will encroach further into the planned movement path of the C-arm. The prediction of the movement of the arm is here indicated by a dashed section O′ of the arm. According to the embodiment of FIG. 5 an avoidance path will in particular be predicted in a preemptive manner. The predicted avoidance path is here again indicated by a corresponding path B′. In FIG. 5 the risk of a collision is still below a predefined threshold value, so that although the avoidance movement B′ is held available, it is not performed. Only if the arm of the patient encroaches into the spatial region R, is the avoidance movement held available performed, in an analogous manner to the scenario shown in FIG. 4.

### LITERATURE

[1] German Patent Application DE 10 2008 046 344.2
[2] German Patent Application DE 10 2008 046 346.9
[3] German Patent Application DE 10 2008 046 348.5
[4] German Patent Application DE 10 2008 046 345.0
[5] Thomas Wösch, “Interaktive movementsführung of a robotann in Alltagsumgebungen durch Kombination von planenden and reaktiven Komponenten” (Interactive Motion Guidance of a Robot Arm through a Combination of Planning-oriented and Reactive Components, Dissertation, TU Graz, 2003.

1.-15. (canceled)

16. A method for monitoring a spatial environment of a mobile device, comprising:
capturing a three-dimensional spatial region of the spatial environment by a detection device during a movement of the device along a predefined path;
creating a three-dimensional environment model from the captured spatial region that specifies a spatial volume in the spatial region occupied by an object;
determining an action of the device for preventing a collision with the object and a risk of the collision based on the environment model and the predefined path; and
performing the action by the device if the risk of the collision exceeds a predefined value.

17. The method as claimed in claim 16,
wherein a model of a maneuvering room of the device is created according to the three-dimensional environment model that specifies the spatial volume in the spatial region in which the device can move safe from the collision, and
wherein the action is determined based on the model of the maneuvering room and a prescribed dynamic model of the movement of the device.

18. The method as claimed in claim 16, wherein the action specifies stopping and/or avoidance of the movement of the device.

19. The method as claimed in claim 16,
wherein a plurality of actions for preventing the collision with the object are determined,
wherein risks of damage upon execution of the actions are determined, and
wherein an action with a lowest risk of damage is performed if the risk of the collision exceeds the predefined value.

20. The method as claimed in claim 19,
wherein the risks of damage are determined depending upon whether the actions cause a personal injury or a material damage, and
wherein the personal injury results in a higher risk of damage than the material damage.

21. The method as claimed in claim 16, wherein the action is determined at least partially based on a prediction of a movement of the object.

22. The method as claimed in claim 16, wherein the spatial region is captured at least partially by the detection device that is arranged on the device and moves with the device.

23. The method as claimed in claim 16, wherein the spatial region is captured at least partially by the detection device that is fixedly arranged and does not move with the device.

24. The method as claimed in claim 16, wherein the spatial region is captured by an active distance-measuring detection device.

25. The method as claimed in claim 24, wherein the active distance-measuring detection device comprises a 3D camera and/or a pivotable laser scanner.

26. The method as claimed in claim 16, wherein a spatial volume occupied by the device is specified in the environment mode in addition to the spatial volume occupied by the object.

27. The method as claimed in claim 16, wherein the mobile device is a medical device and the object is a patient to be examined or treated with the medical device and/or further person captured by the detection device in the spatial region.

28. The method as claimed in claim 16, wherein the mobile device is a C-ain X-ray system.

29. The method as claimed in claim 28, wherein the detection device is attached to the C-arm for capturing a spatial region within the C-arm and/or a spatial region at opposite ends of limbs of the C-arm and/or a spatial region in a spatial direction perpendicular to a plane of the C-arm.

30. The method as claimed in claim 16, wherein the three-dimensional environment model is created and/or updated from the spatial region at cyclical intervals.

31. A device for monitoring a spatial environment of a mobile device, comprising:
a detection device that captures a three-dimensional spatial region of the spatial environment during a movement of the device along a predefined path; and
an analysis device that:
creates a three-dimensional environment model from the captured spatial region that specifies a spatial volume in the spatial region occupied by an object,
determines an action of the device for preventing a collision with the object and a risk of the collision based on the environment model and the predefined path, and
outputs a signal to the device for performing the action if the risk of the collision exceeds a predefined value.
